(19) United States
(12) Patent Application Publication
(10) Pub. No.: US 2012/0022570 A1
(43) Pub. Date: Jan. 26, 2012

(54) DILATION APPARATUS AND METHOD

(75) Inventors: David A. Mire, Cordova, TN (US); Kelli N. Sebastian, Arlington, TN (US); Paul F. Wheeler, Hernando, MS (US)

(73) Assignee: WARSAW ORTHOPEDIC, INC., Warsaw, IN (US)

(21) Appl. No.: 12/842,092

(22) Filed: Jul. 23, 2010

(51) Int. Cl. A61M 29/00

(52) U.S. Cl. 606/191

(57) ABSTRACT

A surgical dilator and method of dilating an incision in a patient is disclosed. The surgical dilator includes a first dilation member having a distal end and a proximal end. The proximal end includes an aperture having a first retaining pin positioned therein that is exposed on an outside surface of the proximal end of the first dilation member. A second dilation member is included that has a distal end, a proximal end, and a hollow interior. The distal end is sized to receive the proximal end of the first dilation member. The second dilation member includes a first compression slot sized to receive the retaining pin of the first dilation member. The first and second dilation members are configured to be oriented in an expanded state and a compressed state in which the second dilation member is compressed down onto the first dilation member such that the first dilation member is received in the hollow interior of the second dilation member.

### BACKGROUND

The present invention relates generally to percutaneous surgeries and more particularly, to devices, methods and systems for performing percutaneous, minimally invasive spinal surgeries.

Traditional surgical procedures for pathologies located deep within the body can cause significant trauma to the intervening tissues. These open procedures often require a long incision, extensive muscle stripping, prolonged retraction of tissues, denervation and devascularization of tissue. Most of these surgeries require a recovery room time of several hours and several weeks of post-operative recovery time due to the use of general anesthesia and the destruction of tissue during the surgical procedure. In some cases, these invasive procedures lead to permanent scarring and pain that can be more severe than the pain leading to the surgical intervention.

Minimally invasive alternatives such as arthroscopic techniques reduce pain, post-operative recovery time and the destruction of healthy tissue. Orthopedic surgical patients have particularly benefited from minimally invasive surgical techniques. The site of pathology is accessed through portals rather than through a significant incision thus preserving the integrity of the intervening tissues. In some instances, these minimally invasive techniques require only local anesthesia. The avoidance of general anesthesia reduces post-operative recovery time and the risk of complications.

Minimally invasive surgical techniques are particularly desirable for spinal and neurosurgical applications because of the need for access to locations deep within the body and the danger of damage to vital intervening tissues. For example, a common open procedure for disc herniation, laminectomy followed by discectomy requires stripping or dissection of the major muscles of the back to expose the spine. In a posterior approach, tissue including spinal nerves and blood vessels around the dural sac, ligaments and muscle must be retracted to clear a channel from the skin to the disc. These procedures normally take at least one-two hours to perform under general anesthesia and require post-operative recovery periods of at least several weeks. In addition to the long recovery time, the destruction of tissue is a major disadvantage of open spinal procedures. This aspect of open procedures is even more invasive when the discectomy is accompanied by fusion of the adjacent vertebrae. Many patients are reluctant to seek surgery as a solution to pain caused by herniated discs and other spinal conditions because of the severe pain sometimes associated with the muscle dissection.

In order to reduce the post-operative recovery time and pain associated with spinal and other procedures, micro-surgical techniques have been developed. The objective of any minimally invasive procedure is to accomplish the same clinical objectives as the traditional, open surgery while minimizing soft tissue retraction. Existing sequential dilation processes consist of inserting multiple increasing diameter dilators until the correct diameter is achieved. A tubular retractor is then placed over the dilators and the dilators are then removed. The retractor is left in place with the surrounding muscle and tissue having been dilated out of the working space.

For some applications, it would be beneficial to be able to dilate an incision quickly without the use of multiple individual dilators. As such, a need exists for a device that will allow physicians to quickly dilate an incision without the use of multiple individual components.

### SUMMARY

According to one aspect a surgical dilator is disclosed that is capable of dilating an incision in a patient. The surgical dilator includes a first dilation member having a distal end and a proximal end. The proximal end includes an aperture having a retaining pin positioned therein that is exposed on an outside surface of the proximal end of the first dilation member. A second dilation member is included that has a second distal end, a second proximal end, and a hollow interior. The second distal end is sized to receive the proximal end of the first dilation member. The second dilation member includes a first L-shaped compression slot that is sized to receive the retaining pin of the first dilation member. The first and second dilation members are configured to be oriented in an expanded state and a compressed state in which the second dilation member is compressed down onto the first dilation member such that the first dilation member is received in the hollow interior of the second dilation member.

In one form, the compression slot comprises a horizontal slot transitioning to a vertical slot. In the expanded state the retaining pin is positioned in the horizontal slot thereby locking the surgical dilator in the expanded state. The second dilation member is operable to be rotated about the first dilation member to orient the retaining pin in the vertical slot thereby allowing the second dilation member to be compressed down onto the first dilation member. As such, the second dilation member is allowed to be inserted into an incision of a patient and encapsulates the first dilation member.

In another representative form, the surgical dilator includes a third dilation member having a third distal end, a third proximal end, and a second hollow interior. The third distal end is sized to receive the second proximal end of the second dilation member. The third dilation member includes a second L-shaped compression slot that is sized to receive a second retaining pin positioned in a second aperture located in the second proximal end of the second dilation member. The third dilation member is operable to be compressed down onto the second dilation member such that the second dilation member is received in the second hollow interior of the third dilation member.

According to another aspect a surgical dilator is disclosed that is capable of dilating an incision in a patient. The surgical dilator includes a dilation member having a first proximal end and a first distal end. The first proximal end includes a retaining pin positioned in a first aperture. A tubular dilation member is included that has a second proximal end tapering downwardly toward a second distal end. The tubular dilation member includes a horizontal slot transitioning into a vertical slot. The dilation member is positioned inside the tubular dilation member such that the retaining pin is operable to travel in the horizontal and vertical slots. When the retaining pin is positioned in the horizontal slot the surgical dilator is in an expanded state and as the tubular dilation member is rotated to expose the retaining pin to the vertical slot the tubular dilation member is operable to be compressed down onto the dilation member as the retaining pin travels in the vertical slot.

In one representative form, the first proximal end includes a first portion having a generally uniform circular cross-section and said distal end includes a second portion that tapers downwardly toward an insertion tip. When the retaining pin is positioned in the horizontal slot the tubular dilation member is prevented from being compressed down onto the dilation member. An end of the vertical slot prevents the tubular dilation member from being further compressed down onto the dilation member when the retaining pin makes contact with the end.

In yet another representative form, an aperture is located in the second proximal end of the tubular dilation member that includes a second retaining pin positioned therein. In this form, a second tubular dilation member is included that has a third proximal end tapering downwardly toward a third distal end. The second tubular dilation member includes a second horizontal slot transitioning to a second vertical slot. The second dilation member is positioned inside the tubular dilation member such that the second retaining pin is operable to travel in the second horizontal and vertical slots. When the second retaining pin is positioned in the second horizontal slot the second tubular dilation member is prevented from being compressed down onto the tubular dilation member and as the second tubular dilation member is rotated to expose the second retaining pin to the second vertical slot the second tubular dilation member is operable to be compressed down onto the tubular dilation member as the second retaining pin travels in the second vertical slot.

Another aspect of the present invention discloses a method of dilating an incision in a patient. The method includes the steps of inserting a distal end of a first dilation member into the incision; pressing the first dilation member down into the incision until reaching a proximal end of the first dilation member; rotating a second dilation member connected to the proximal end of the first dilation member to expose a retaining pin in the first dilation member to a vertical slot in the second dilation member; and compressing the second dilation member down onto the first dilation member and into the incision.

In one form, the method of dilating the incision can also include the step of rotating a third dilation member connected to a proximal end of the second dilation member to expose a second retaining pin in the second dilation member to a second vertical slot in the third dilation member. The third dilation member can then be compressed down onto the second dilation member and into the incision. In alternative forms, the method can also include stimulating a tip of said first dilation member with an electrical signal.

Related features, aspects, embodiments, objects and advantages of the present invention will be apparent from the following description.

### BRIEF DESCRIPTION OF THE DRAWINGS

FIG. 1 is a perspective view of a representative surgical dilator.

FIG. 2 is another perspective view of the surgical dilator illustrated in FIG. 1.

FIG. 3 is a perspective view of a dilation member of the surgical dilator illustrated in FIG. 1.

FIG. 4 is a perspective view of a second dilation member of the surgical dilator illustrated in FIG. 1.

FIG. 5 is an end view of the second dilation member illustrated in FIG. 4.

FIG. 6 is a perspective view of a portion of the surgical dilator illustrated in FIG. 1.

FIG. 7 is a perspective view of another representative surgical dilator.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

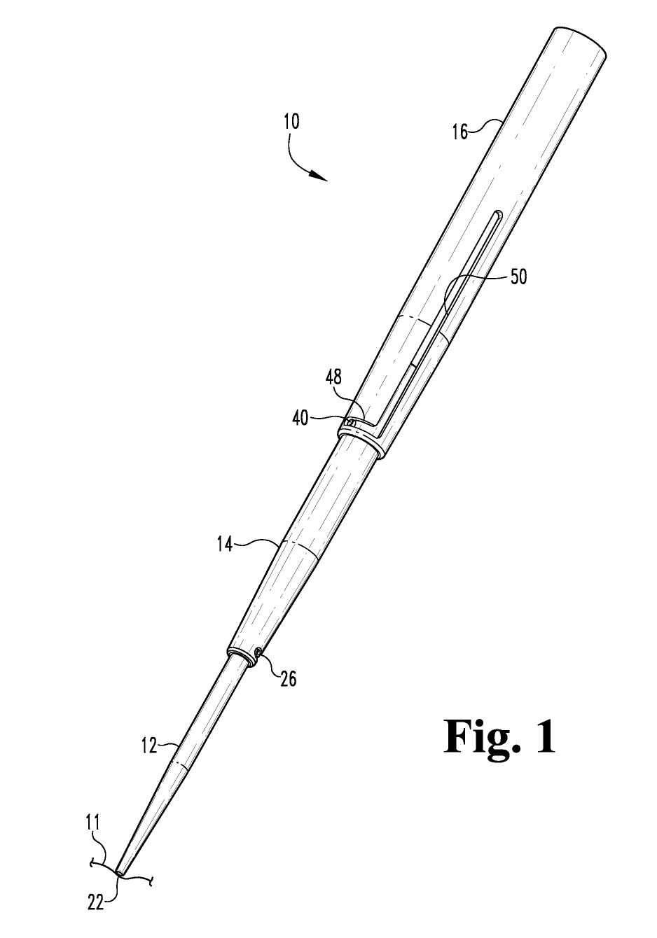

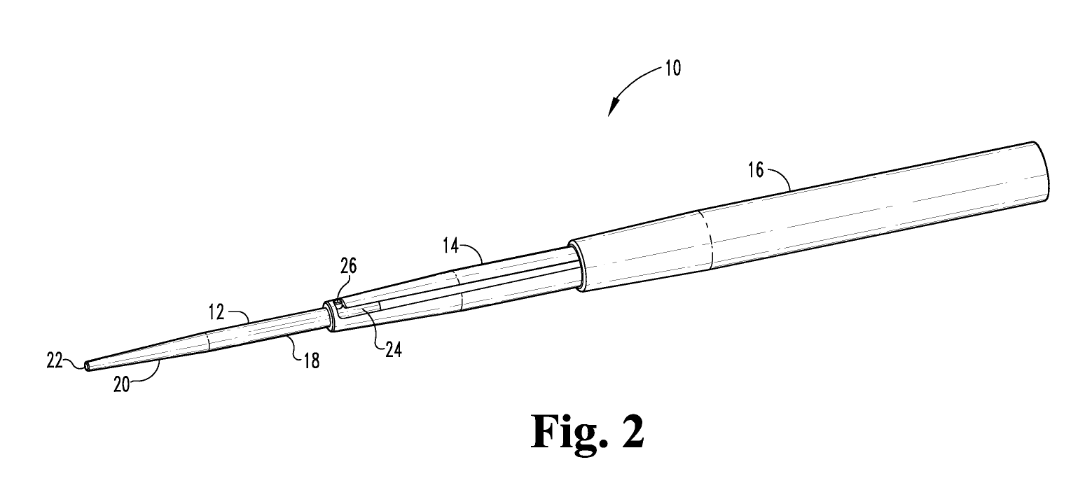

Referring collectively to FIGS. 1 and 2, a soft tissue surgical dilator 10 is disclosed that comprises a series of at least two or more interlocked dilation members that compress upon themselves when inserted into a patient. As illustrated, prior to insertion into an incision 11 in a patient to be dilated, the dilator 10 is oriented in an expanded state. As the dilator 10 is inserted into the patient each interlocked dilation member is configured to be unlocked from the dilation member already inserted so that the unlocked dilation member can be compressed down on the previous dilation member and inserted into the patient. As the dilation member is compressed downward into the incision 11, the previous dilation member already inserted moves into an interior cavity defined in the dilation member being inserted.

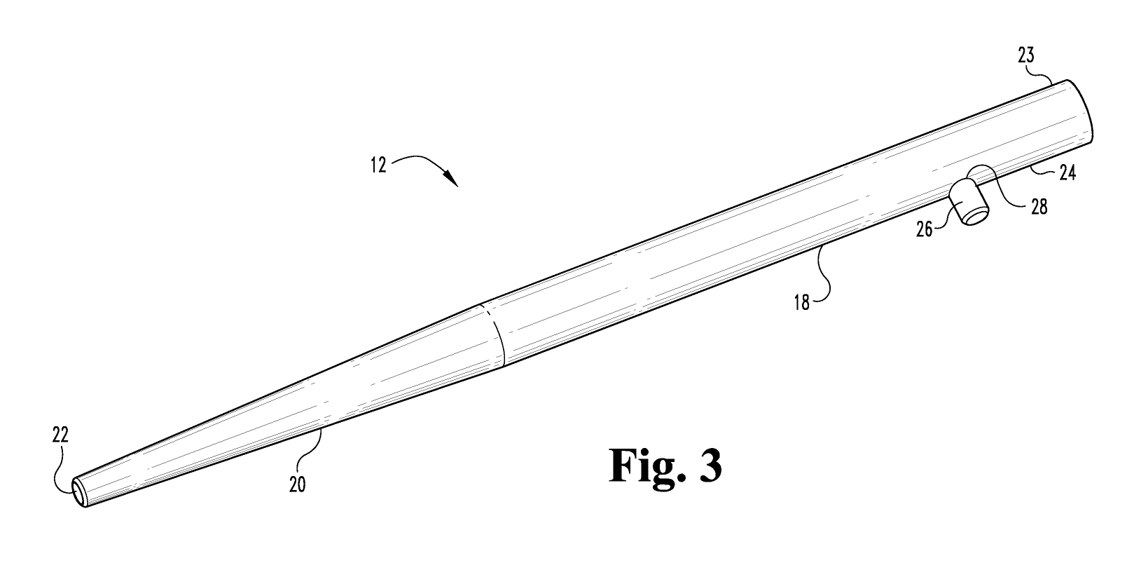

The dilator 10 includes a first dilation member 12, a second tubular dilation member 14, and a third tubular dilation member 16. In one form, the first dilation member 12 comprises a body having a generally circular cross-section shaped upper portion 18 that transitions into a generally tapered or conically shaped lower portion 20 that terminates at an insertion tip 22. The insertion tip 22 can be rounded, tapered, pointed, or flat to aid in the insertion of the first dilation member 12 into the incision 11. The tapered lower portion 20 also has a generally circular cross-section shape. In other forms, the first dilation member 12 can taper downwardly from a proximal end 23 to the insertion tip 22. See FIG. 3. As the first dilation member 12 is inserted into the incision 11, the tapered portions cause the incision to expand thereby dilating the incision 11.

The first dilation member 12 can be a solid structure in some forms and a tubular structure in other forms that allows a guide wire to be inserted into the interior passage defined by the dilation members 12, 14, 16. At approximately a proximal end 24 of the first dilation member 12 a retaining pin 26 is positioned or inserted into an aperture 28 in the first dilation member 12. See FIG. 3. As set forth in greater detail below, the retaining pin 26 is used to movably secure or interlock the first dilation member 12 within the second dilation member 14. The retaining pin 26 is inserted into the aperture 28 such that no surfaces of the retaining pin 26 are exposed beyond any outer surface of the second dilation member 14. Although the dilation members disclosed herein are illustrated as having generally circular cross-section shapes, other shapes can be utilized in other forms so long as the dilation members are capable of being compressed down onto one another.

Referring to FIGS. 2, 4 and 5, the second dilation member 14 comprises a tubular body having a generally circular cross-section shape. A hollow interior 29 is defined in the second dilation member 14 that is sized and configured to receive the first dilation member 12 as the second dilation member 14 is compressed down onto the first dilation member 12. A distal end 30 of the second dilation member 14 includes a horizontal or longitudinal slot 32 that transitions to a vertical or latitudinal slot 34 that travels upwardly to approximately a proximal end 36 of the second dilation member 14. The horizontal and vertical slots 32, 34, which can be collectively referred to as a compression slot, are sized and configured such that the retaining pin 26 of the first dilation member 12 is allowed to travel within the slots 32, 34. In the expanded state, the horizontal slot 32 prevents the second dilation member 14 from being compressed down on the first dilation member 12. Once the first dilation member 12 is properly positioned in the incision 11 in the patient, the second dilation member 14 is rotated about a horizontal or longitudinal axis to expose the retaining pin 26 of the first dilation member 12 to the vertical slot 34. The second dilation member 14 is then permitted to be compressed onto the first dilation member 12 as the retaining pin 26 travels upwardly in the vertical slot 34. Once the retaining pin 26 reaches the end of the vertical slot 34, the second dilation member 14 is prevented from being further compressed down onto the first dilation member 12.

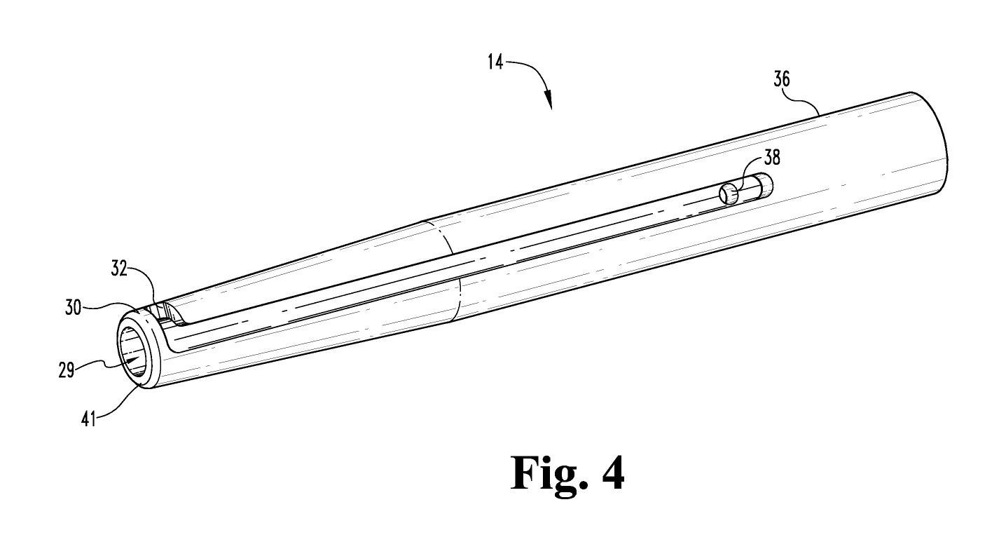

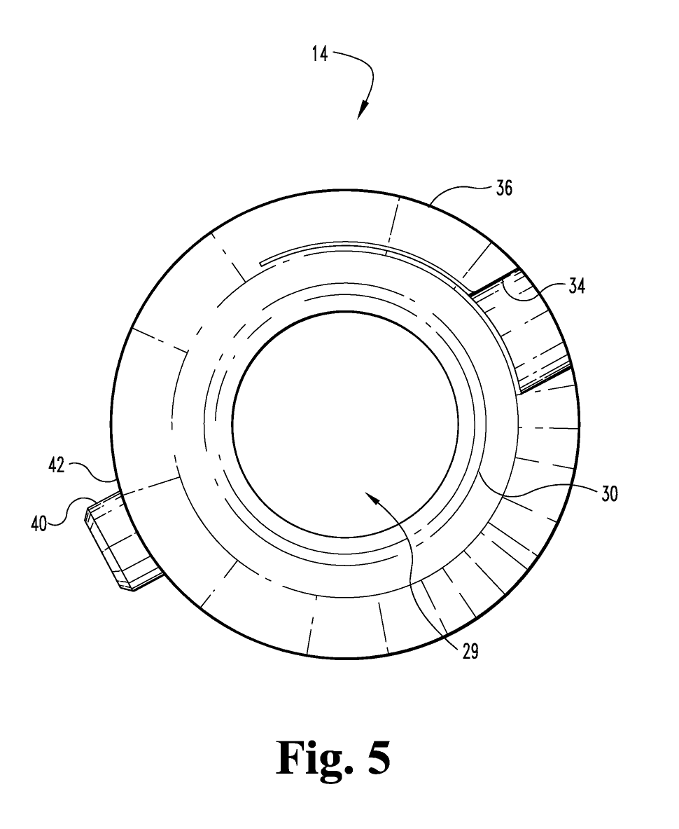

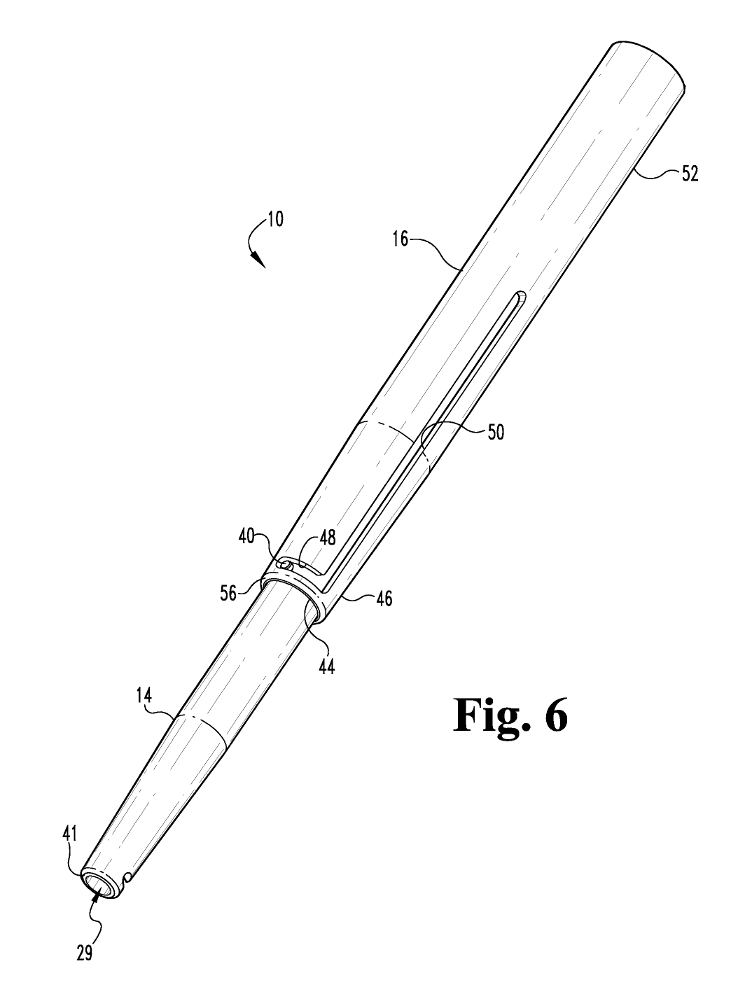

As illustrated in FIG. 4, the proximal end 36 of the second dilation member 14 includes an aperture 38 located on a respective side of the tubular body. Referring to FIGS. 5 and 6, the aperture 38 is sized and configured to receive a second retaining pin 40. The second retaining pin 40 extends outwardly from a side surface 42 of the second dilation member 14, but not far enough to be exposed beyond an outside surface of the third dilation member 16. In one form, the proximal end 36 of the second dilation member 14 has a larger outside diameter than the distal end 30. Thus, the second dilation member 14 comprises a tubular body that tapers downwardly from the proximal end 36 to the distal end 30. As the second dilation member 14 is inserted into the incision 11, the taper causes the incision 11 to expand thereby further dilating the incision 11. An end 41 of the second dilation member 12 is tapered or rounded to aid in the insertion of the second dilation member 12 into the incision 11.

Referring to FIG. 6, the third dilation member 16 also comprises a tubular body that defines a hollow interior 44 that is sized and configured to receive the second dilation member 14. A distal end 46 of the third dilation member 16 includes a horizontal or longitudinal slot 48 that transitions to a vertical or latitudinal slot 50 that travels upwardly a predetermined distance toward a proximal end 52 of the third dilation member 14. The horizontal and vertical slots 48, 50 can be referred to as a compression slot. Once the second dilation member 14 is inserted into the hollow interior 44 of the third dilation member 16, the retaining pin 40 is inserted into the aperture 38 in the second dilation member 14 thereby movably interlocking the second dilation member 14 within the third dilation member 16. As with the first and second dilation members 12, 14, the third dilation member 16 tapers downwardly from the proximal end 52 to the distal end 46. As the third dilation member 16 is inserted in the incision 11 and compressed down onto the second dilation member 14, the incision 11 further expands as a function of the outside diameter of the third dilation member 16. An end 56 of the third dilation member 14 includes a tapered or rounded portion that assists in the insertion of the third dilation member 14 into the incision 11.

As described with respect to the first and second dilation members 12, 14, after the second dilation member 14 has been inserted into the incision 11 such that the incision 11 reaches approximately the end 56 of the third dilation member 16, the surgeon can then rotate the third dilation member 16 to expose the retaining pin 40 to the vertical slot 50. The third dilation member 16 can then be compressed down onto the second dilation member 14 and into the incision 11 in the patient. As the third dilation member 16 is compressed down, the second dilation member 14 travels up into the hollow interior 44 of the third dilation member 16. Once the retaining pin 40 in the second dilation member 14 reaches the end of the vertical slot 50, the third dilation member 16 is prevented from being further compressed down onto the second dilation member 14.

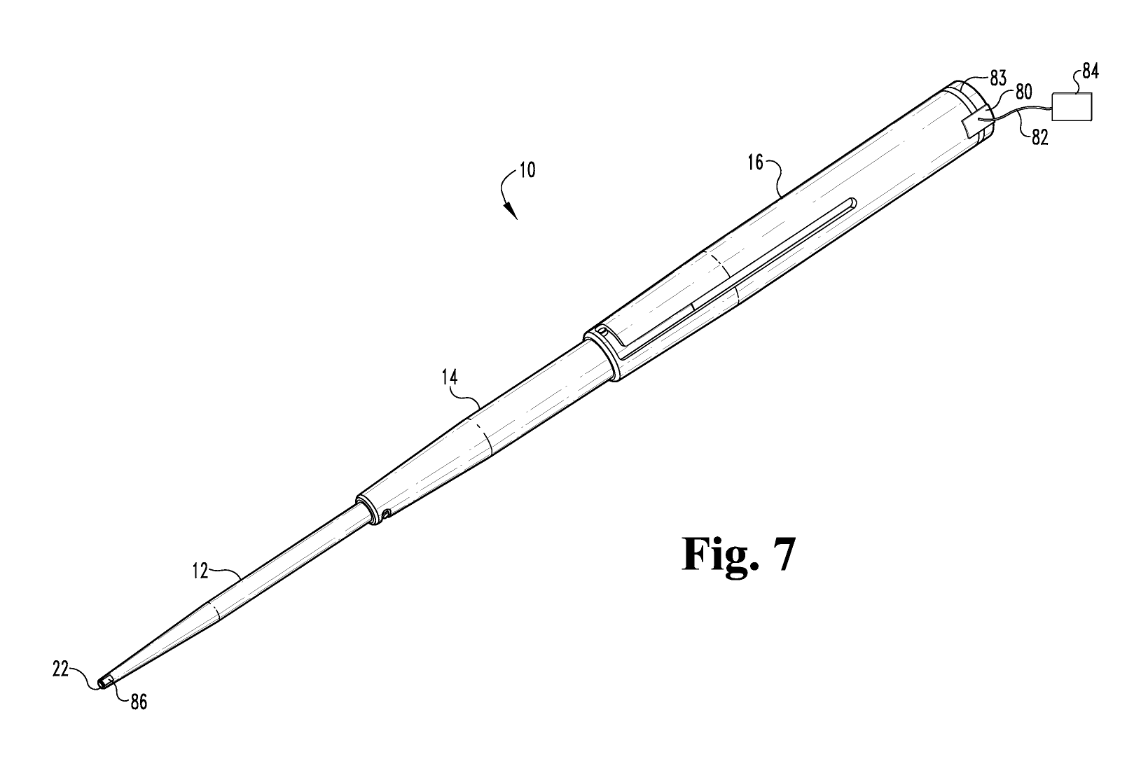

Referring to FIG. 7, in one representative form of the present invention the dilator 10 is provided with neuromonitoring capabilities. In this form, the dilator 10 can be substantially formed from a non-conductive material such as, for example, anodized aluminum. In this form, a portion of the third dilation member 16 includes an area 80 that is conductive and exposed on an outer surface of the third dilation member 16. The conductive area 80 permits the attachment of a stimulation signal transfer device 82 such as, for example, one or more wires to the third dilation member 16. The stimulation signal transfer device 82 could be attached by way of a clip 83 or some other similar attachment device. The stimulation signal transfer device 82 is connected with an electric stimulation signal generator 84 that is operable to stimulate the dilator 10. The tip 22 of the first dilation member 12 also includes a conductive area 86 exposed on an outer surface of the first dilation member 12. Neuromonitoring capability is achieved by stimulating the tip 22 with electric signals, via the conductive area 86, to aid in detecting the proximity of the tip 22 to any neural structures. In other forms, the second dilation member 14 could include the conductive area 80 and conductive area 86.

Another aspect of the present invention discloses a method of dilating an incision 11 in a patient. The method includes the step of inserting a distal end 20 of a first dilation member 12 into an incision 11. The first dilation member 12 is then pressed down into the incision 11 until reaching a proximal end 18 of the first dilation member 12. A second dilation member 14 is then rotated to expose a retaining pin 26 in the first dilation member 12 to a vertical slot 34 in the second dilation member 14. The second dilation member 14 is then compressed down onto the first dilation member 12 and into the incision 11. A third dilation member 16 is then rotated to expose a second retaining pin 40 in the second dilation member 14 to a second vertical slot 50 in the third dilation member 16. The third dilation member 16 is then compressed down onto the second dilation member 14 and into the incision 11. In some forms, during the dilation process, a tip 22 of the first dilation member 12 is stimulated with an electrical signal to provide a neuromonitoring capability.

Although the dilators disclosed herein have been illustrated as having three dilation members, it should be appreciated that two or more (e.g.—four, five, and so forth) dilation members can be used in other forms of the present invention. In one form, the surgical dilators disclosed herein are sized and configured to achieve a range of dilation from approximately 5.3 mm to 21 mm, but other ranges are envisioned. The dilation members and retaining pins disclosed herein can be manufactured from various materials such as aluminum, anodized aluminum, plastic, titanium, titanium alloys, steel, and so forth.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “proximal” and “distal” refer to the direction closer to and away from, respectively, an operator (e.g., surgeon, physician, nurse, technician, etc.) who would insert the medical implant and/or instruments into the patient. For example, the portion of a medical instrument first inserted inside the patient's body would be the distal portion, while the opposite portion of the medical device (e.g., the portion of the medical device closest to the operator) would be the proximal portion.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. A surgical dilator, comprising:
a first dilation member having a first distal end and a first proximal end, wherein said first proximal end includes a first aperture having a retaining pin positioned therein that is exposed on an outside surface of said first proximal end of said first dilation member; and
a second dilation member having a second distal end, a second proximal end, and a hollow interior, said second distal end being sized to receive said first proximal end of said first dilation member, said second dilation member including a first compression slot sized to receive said first retaining pin of said first dilation member, said first and second dilation members being configured to be oriented in an expanded state and a compressed state in which said second dilation member is compressed down onto said first dilation member such that said first dilation member is received in said hollow interior of said second dilation member.

2. The surgical dilator of claim 1, wherein said compression slot comprises a horizontal slot transitioning to a vertical slot.

3. The surgical dilator of claim 2, wherein in said expanded state said retaining pin is positioned in said horizontal slot thereby locking said surgical dilator in said expanded state.

4. The surgical dilator of claim 3, wherein said second dilation member is operable to be rotated about said first dilation member to orient said retaining pin in said vertical slot thereby allowing said second dilation member to be compressed down onto said first dilation member.

5. The surgical dilator of claim 1, further comprising a third dilation member having a third distal end, a third proximal end, and a second hollow interior, said third distal end being sized to receive said second proximal end of said second dilation member, said third dilation member including a second compression slot sized to receive a second retaining pin positioned in a second aperture located in said second proximal end of said second dilation member.

6. The surgical dilator of claim 5, wherein said third dilation member is operable to be compressed down onto said second dilation member such that said second dilation member is received in said second hollow interior of said third dilation member.

7. The surgical dilator of claim 1, wherein a tip of said first dilation member includes a portion made from a conductive material and an upper portion of said second dilation member includes a second portion made from a conductive material such that a stimulator can be connected with said second portion to transfer a stimulation signal to said first portion.

8. A surgical dilator, comprising:
a dilation member having a first proximal end and a first distal end, wherein said first proximal end includes a retaining pin positioned in a first aperture; and
a tubular dilation member having a second proximal end tapering downwardly toward a second distal end, wherein said tubular dilation member includes a horizontal slot transitioning into a vertical slot, wherein said dilation member is positioned inside said tubular dilation member such that said retaining pin is operable to travel in said horizontal and vertical slots, wherein when said retaining pin is positioned in said horizontal slot said surgical dilator is in an expanded state and as said tubular dilation member is rotated to expose said retaining pin to said vertical slot said tubular dilation member is operable to be compressed down onto said dilation member as said retaining pin travels in said vertical slot.

9. The surgical dilator of claim 8, wherein said first proximal end includes a first portion having a generally uniform circular cross-section and said distal end includes a second portion that tapers downwardly toward an insertion tip.

10. The surgical dilator of claim 8, wherein when said retaining pin is positioned in said horizontal slot said tubular dilation member is prevented from being compressed down onto said dilation member.

11. The surgical dilator of claim 8, wherein an end of said vertical slot prevents said tubular dilation member from being further compressed down onto said dilation member when said retaining pin makes contact with said end.

12. The surgical dilator of claim 8, further comprising an aperture in said second proximal end of said tubular dilation member having a second retaining pin positioned therein.

13. The surgical dilator of claim 12, further comprising a second tubular dilation member having a third proximal end tapering downwardly toward a third distal end, wherein said second tubular dilation member includes a second horizontal slot transitioning to a second vertical slot, wherein said second dilation member is positioned inside said tubular dilation member such that said second retaining pin is operable to travel in said second horizontal and vertical slots, wherein when said second retaining pin is positioned in said second horizontal slot said second tubular dilation member is prevented from being compressed down onto said tubular dilation member and as said second tubular dilation member is rotated to expose said second retaining pin to said second vertical slot said second tubular dilation member is operable to be compressed down onto said tubular dilation member as said second retaining pin travels in said second vertical slot.

14. The surgical dilator of claim 13, wherein an insertion end of said second tubular dilation member is tapered downwardly.

15. The surgical dilator of claim 8, wherein an insertion end of said tubular dilation member is tapered downwardly.

16. The surgical dilator of claim 8, wherein a tip of said dilation member includes a portion made from a conductive material and an upper portion of said tubular dilation member includes a second portion made from a conductive material such that a stimulator can be connected with said second portion to transfer a stimulation signal to said first portion.

17. A method of dilating an incision, comprising:
inserting a distal end of a first dilation member into said incision;
pressing said first dilation member down into said incision until reaching a proximal end of said first dilation member;
rotating a second dilation member connected to said proximal end of said first dilation member to expose a retaining pin in said first dilation member to a vertical slot in said second dilation member; and
compressing said second dilation member down onto said first dilation member and into said incision.

18. The method of claim 17, further comprising rotating a third dilation member connected to a proximal end of said second dilation member to expose a second retaining pin in said second dilation member to a second vertical slot in said third dilation member.

19. The method of claim 18, further comprising compressing said third dilation member down onto said second dilation member and into said incision.

20. The method of claim 17, further comprising stimulating a tip of said first dilation member with an electrical signal.
